(19) United States
(12) Patent Application Publication
(10) Pub. No.: US 2024/0325706 A1
(43) Pub. Date: Oct. 3, 2024

(54) REDUCIBLE DIAMETER SHEATH

(71) Applicant: Boston Scientific Scimed, Inc., Maple Grove, MN (US)

(72) Inventors: Daniel Presteng, Corcoran, MN (US); Benjamin Jerome Bidne, Loretto, MN (US); Lauren Ann Sandy, Minneapolis, MN (US)

(73) Assignee: Boston Scientific Scimed, Inc., Maple Grove, MN (US)

(21) Appl. No.: 18/621,586

(22) Filed: Mar. 29, 2024

Related U.S. Application Data
(60) Provisional application No. 63/456,035, filed on Mar. 31, 2023.

(51) Int. Cl. A61M 39/06; A61M 39/02; A61M 60/117

(52) U.S. Cl. CPC A61M 39/0613; A61M 60/117; A61M 2039/0258; A61M 2039/0273; A61M 2039/0673; A61M 2205/0216

(57) ABSTRACT

A sheath with an expandable and reducible diameter includes a body having a lumen and that is movable between a first configuration and a second configuration. The body has a first diameter size in the first configuration that is smaller than a second diameter size in the second configuration. The body includes a shaft made from a first material and a wire made from a second material.

### CROSS REFERENCE TO RELATED APPLICATIONS

This application claims the benefit of U.S. Provisional Patent Application Ser. No. 63/456,035, filed Mar. 31, 2023, which is incorporated herein by reference.

### TECHNICAL FIELD

The present disclosure relates to sheaths for delivering a medical device, and more particularly, sheaths that are capable of reducing and increasing their size in a radial direction.

### BACKGROUND

When inserting percutaneous circulatory devices or various other implantable medical devices, such devices may be inserted into a blood vessel through an introducer sheath, which is inserted at an access site and into the blood vessel. After placement of the device within the body, the introducer sheath may be removed and replaced with a sheath of a smaller diameter to increase blood flow through the blood vessel and thereby reduce ischemia issues for the patient.

### SUMMARY

In Example 1, a sheath with an expandable diameter includes a body having a lumen and that is movable between a first configuration and a second configuration. The body has a first inner diameter size in the first configuration that is smaller than a second inner diameter size in the second configuration. The body includes a shaft made from a first material and a wire made from a second material.

In Example 2, the sheath of Example 1, further including: a hub coupled to the body, and an actuator coupled to the hub. Upon actuation, the actuator is configured to cause the body to move between the first configuration and the second configuration.

In Example 3, the sheath of Example 2, wherein—upon actuation—the actuator is configured to pull the wire into the hub to cause the body to move from the second inner diameter size to the first inner diameter size.

In Example 4, the sheath of Example 2 or Example 3, wherein the actuator is a switch movable within a slot in the hub.

In Example 5, the sheath of any of the preceding Examples, wherein the wire is helical shaped.

In Example 6, the sheath of any of the preceding Examples, wherein the wire is coupled to or integral with wing portions that are collapsible in response to the wire being pulled.

In Example 7, the sheath of any of the preceding Examples, wherein the wire is embedded in the shaft.

In Example 8, the sheath of any of the preceding Examples, wherein the first material comprises an elastomer, wherein the second material comprises a metal.

In Example 9, the sheath of any of the preceding Examples, wherein the first material comprises a thermoplastic polyurethane elastomer.

In Example 10, the sheath of any of the preceding Examples, wherein the body expands to the second diameter size as a medical device is inserted through the lumen.

In Example 11, the sheath of Example 10, wherein the wire is arranged to collapse before and after insertion of the medical device to collapse the body to the first diameter size.

In Example 12, the sheath of any of Examples 1-9, further including a support member coupled to or formed as part of the body and extending along a longitudinal axis of the body.

In Example 13, the sheath of Example 12, further including wing portions positioned between the wire and the support member.

In Example 14, the sheath of any of the preceding Examples, wherein the first material has a lower durometer value compared to the second material.

In Example 15, the sheath of any of the preceding Examples, wherein the first material has a Shore hardness value from 70A to 80A.

In Example 16, a sheath is movable between a collapsed diameter and an expanded diameter and includes a body having a lumen and that is movable between a first configuration and a second configuration. The body has a first diameter size in the first configuration that is smaller than a second diameter size in the second configuration. The body includes a shaft made from a first material and a wire comprising a second material.

In Example 17, the sheath of Example 16, further including: a hub coupled to the body, and an actuator coupled to the hub. Upon actuation, the actuator is configured to cause the body to move between the first configuration and the second configuration.

In Example 18, the sheath of Example 17, wherein—upon actuation—the actuator is configured to pull the wire into the hub to cause the body to move from the second diameter size to the first diameter size.

In Example 19, the sheath of Example 18, wherein the actuator is a switch movable within a slot in the hub.

In Example 20, the sheath of Example 16, wherein the wire is helical shaped.

In Example 21, the sheath of Example 16, wherein the wire is coupled to or integral with wing portions that are collapsible in response to the wire being pulled.

In Example 22, the sheath of Example 16, wherein the wire is embedded in the shaft.

In Example 23, the sheath of Example 16, wherein the first material comprises an elastomer, wherein the second material comprises a metal.

In Example 24, the sheath of Example 16, wherein the first material comprises a thermoplastic polyurethane elastomer.

In Example 25, the sheath of Example 16, further including a support member coupled to or formed as part of the body and extending along a longitudinal axis of the body.

In Example 26, the sheath of Example 25, further including wing portions positioned between the wire and the support member.

In Example 27, the sheath of Example 26, wherein the wing portions and the wire are integrally formed from one part.

In Example 28, the sheath of Example 25, wherein the wing portions are arranged perpendicular to the support member in the second configuration and are arranged at an angle less than 90 degrees in the first configuration.

In Example 29, the sheath of Example 16, wherein the first material has a lower durometer value compared to the second material.

In Example 30, the sheath of Example 16, wherein the body expands to the second diameter size as a medical device is inserted through the lumen.

In Example 31, the sheath of Example 30, wherein the wire is arranged to collapse before and after insertion of the medical device to collapse the body to the first diameter size.

In Example 32, the sheath of Example 16, wherein the first diameter size and the second diameter size are inner diameters of the body defined by the lumen.

In Example 33, the sheath of Example 16, wherein the first diameter size and the second diameter size are outer diameters of the body defined by an outer surface of the body.

In Example 34, a sheath—movable between a collapsed diameter and an expanded diameter—includes a body having a lumen and means for actuating the body between the collapsed diameter and the expanded diameter.

In Example 35, the sheath of Example 34, wherein the means for actuating comprises a wire and an actuator coupled to the wire and configured to pull the wire to actuate the body to the collapsed diameter.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 illustrates a blood vessel and a sheath, in accordance with certain embodiments of the present disclosure.

FIG. 2 illustrates a medical device positioned within the sheath of FIG. 1, in accordance with certain embodiments of the present disclosure.

FIGS. 3A-5B illustrate sheaths capable of reducing and increasing their size in a radial direction, in accordance with certain embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the claims.

### DETAILED DESCRIPTION

An introducer sheath can be used to assist with inserting a medical device such as a percutaneous circulatory device. After positioning a medical device within a patient's body, the introducer sheath may be removed and replaced with a sheath of a smaller diameter to increase blood flow through the blood vessel and thereby reduce ischemia issues for the patient. However, replacement of the introducer sheath may cause additional trauma to the access site of the patient. As such, there remains a need for a single sheath that may be used for delivery of the medical device and for extended periods of time to reduce ischemia issues. Certain embodiments of the present disclosure are accordingly directed to multi-purpose sheaths. More specifically, certain embodiments involve sheaths that are capable of reducing and increasing their size in a radial direction (e.g., increasing or decreasing the size of inner and/or outer diameters).

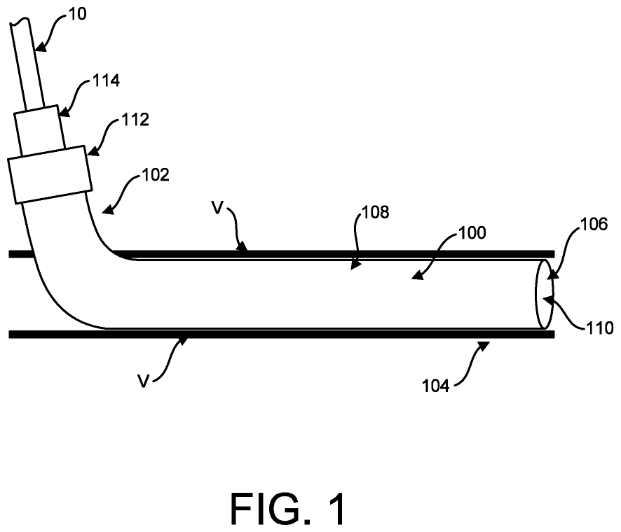

FIG. 1 illustrates a side cross sectional view of a blood vessel V with a sheath 100, inserted at least partially into the blood vessel V. While the disclosure herein is made with reference largely to the sheath 100, the disclosure may also apply to a repositioning sheath. In some embodiments, the sheath 100 is used for facilitating the passage of various relatively large medical devices such as a blood pump through the sheath 100 and into the blood vessel V. As will be described further, the sheath 100 may be used for delivering the medical device into the blood vessel V and may also maintain its positioning within the blood vessel for extended periods of time because of the ability of the sheath 100 to alter its diameter. The sheath 100 comprises a proximal end 102 and a distal end 104 that is opposite the proximal end 102. The sheath 100 includes a proximal opening adjacent the proximal end 102 and a distal opening 106 adjacent the distal end 104. A body portion 108 of the sheath 100 extends between the proximal end 102 and the distal end 106, and the body portion 108 defines a lumen 110 of the sheath 100.

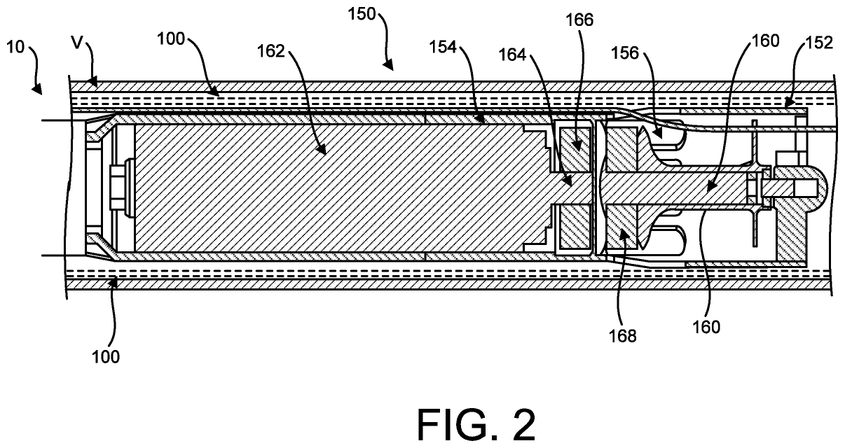

A hub 112 is commonly included at the proximal end 102 and over the proximal opening of the sheath 100. The hub 112, also referred to herein as a hemostasis valve hub, is configured for hemostasis (e.g., to help prevent blood from leaking out of the sheath 100 during use). More specifically, a medical device, for example a catheter 10, may be inserted through the hub 112 and the sheath 100 and into the blood vessel V, and the hub 112 may maintain hemostasis between the catheter 10, the sheath 100, and the external surroundings. In some embodiments, the catheter 10 may couple to another medical device, as the blood pump 150 shown in FIG. 2. After insertion of the catheter 10, fixation of the axial and radial position of the catheter 10 may be desired to ensure that the catheter 10 (and any coupled medical device) remains in the proper position during use. Further, in some instances, it may also be desired for the operator to reposition the catheter 10 (and any coupled medical device) after insertion. As such, the hub 112 may comprise a tightening port 114 composed of several components within the hub 112 that helps fix the catheter 10 with respect to the hub 112 and blood vessel V. The hub 112 and tightening port 114 may also allow for the repositioning of the catheter 10 with respect to the hub 112 and blood vessel V.

FIG. 2 illustrates a cross-sectional view of the sheath 100 (shown in dotted lines) after insertion of a medical device, illustratively a blood pump 150, into the sheath 100. As noted above, in some embodiments a catheter may be coupled to a proximal end of the blood pump 150 and extend outside the blood vessel V and sheath 100. The blood pump 150 generally includes an impeller assembly housing 152 and a motor housing 154. In some embodiments, the impeller assembly housing 152 and the motor housing 154 may be integrally or monolithically constructed. The impeller assembly housing 152 carries an impeller assembly 156 therein. The impeller assembly 156 includes an impeller shaft 158 and an impeller 160 that rotates relative to the impeller assembly housing 152 to drive blood through the blood pump 150. More specifically, the impeller 160 causes blood to flow from a blood inlet through the impeller assembly housing 152 and out of a blood outlet. In some embodiments, the impeller shaft 158 and the impeller 160 may be integrated, and in other embodiments the impeller shaft 158 and the impeller 160 may be separate components. The inlet may be formed on an end portion of the impeller assembly housing 152, and the outlet may be formed on a side portion of the impeller assembly housing 152. In other embodiments, the inlet and/or the outlet may be formed on other portions of the impeller assembly housing 152. In some embodiments, the impeller assembly housing 152 may couple to a distally extending cannula, and the cannula may receive and deliver blood to the inlet.

With continued reference to FIG. 2, the motor housing 154 carries a motor 162, and the motor 162 is configured to rotatably drive the impeller 160 relative to the impeller assembly housing 152. In the illustrated embodiment, the motor 162 rotates a drive shaft 164, which is coupled to a driving magnet 166. Rotation of the driving magnet 166 causes rotation of a driven magnet 168. In embodiments incorporating the impeller shaft 158, the impeller shaft 158 and the impeller 160 are configured to rotate with the driven magnet 168. In other embodiments, the motor 162 may couple to the impeller assembly housing 152 via other components. While the sheath 100 is illustrated above with the use of the blood pump 150, various other medical devices may be used in conjunction with the sheath 100 and the hemostasis valve hub 120.

The remaining Figures illustrate various examples of sheaths that are capable of reducing and increasing the size of their inner and/or outer diameters.

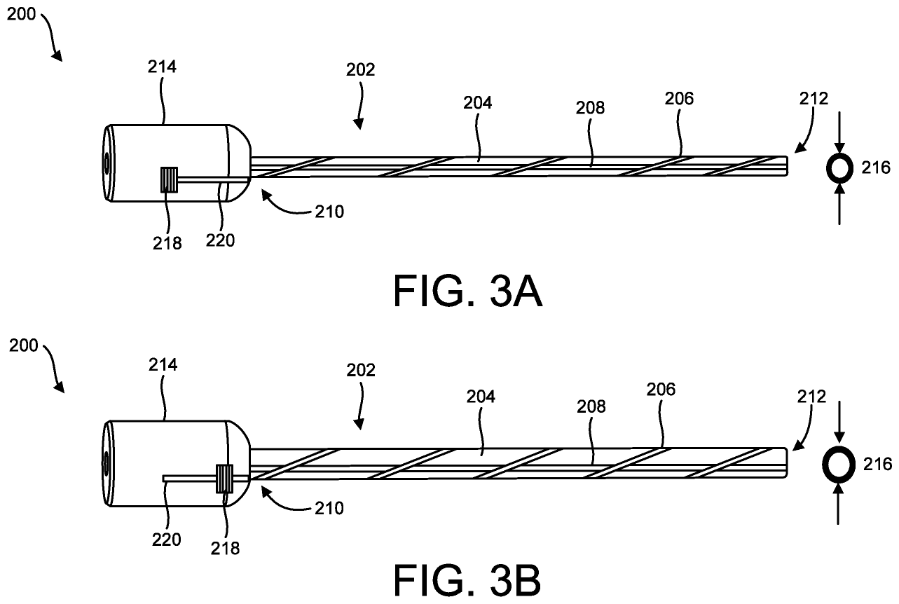

FIGS. 3A and 3B show a sheath 200 in different configurations. FIG. 3A shows the sheath 200 in a first configuration where the size of the outer diameter of the sheath 200 is smaller compared to the size of the outer diameter of the sheath 200 when it is in a second configuration, which is shown in FIG. 3B. In addition to being able to change the size of the outer diameter, the sheath 200 can be controlled to change the size of the inner diameter of the sheath 200 (e.g., the size of the diameter of a lumen within the sheath 200).

The sheath 200 includes a body 202, which includes a shaft 204, one or more wires 206, and one or more support members 208. The shaft 204 extends between a proximal end 210 and a distal end 212, and the shaft 204 is coupled to a hub 214.

In certain embodiments, the wire 206 and/or the support member 208 is embedded in the shaft 204. For example, the shaft 204 could be molded around the wire 206 and/or the support member 208 such that the shaft 204 covers the wire 206 and/or the support member 208. As another example, the shaft 204 could comprise multiple layers (e.g., an inner shaft portion and an outer shaft portion), and the wire 206 and/or the support member 208 could be positioned between the layers.

The shaft 204 can comprise a first material, and the wire 206 and the support member 208 can comprise a second material. In some embodiments, the first material is a softer material (e.g., less rigid material) relative to the second material. For example, the first material may have a lower Shore hardness value such as ranging from 70A to 80A. In certain embodiments, the first material may have a higher flexibility and less resistance to deformation relative to the second material. Further, the first material may have a lower surface roughness relative to the second material such that the first material creates less friction when a medical device is inserted into the body 202. In some embodiments, the first material is or comprises an elastomer material (e.g., thermoplastic elastomer, thermoplastic polyurethane elastomer), and the second material is or comprises a metallic material such as steel (e.g., spring steel) or an alloy comprising nickel and titanium (e.g., nitinol). However, other applicable materials may be used in place of the materials mentioned above or in addition to such materials. In certain embodiments, a coating may be applied to an interior surface of the body 202 to lubricate the interior surface so that less friction is created between the body 202 and a medical device passing through the body 202. In embodiments where the shaft 204 includes multiple layers, the layers can comprise the same or different materials. Further, in certain embodiments, the wire 206 and the support member 208 could comprise different materials than each other.

The body 202 can be cylindrically shaped and have an inner diameter and an outer diameter 216—the size of which can be controlled to be decreased or increased. In the Figures, the outer diameter 216 of the body 202 is smaller in FIG. 3A compared to the outer diameter 216 in FIG. 3B. Further, the inner diameter of the body is smaller in FIG. 3A compared to the inner diameter in FIG. 3B.

The size of the body 202 in a radial direction can be modified using an actuator 218 that is coupled to the hub 214. The actuator 218 can be moved to cause the sheath 200 to switch between configurations. For example, the actuator 218 can be moved to increase or reduce the size of the body 202 as defined by its inner diameter or its outer diameter 216. The actuator 218 may be a button, switch (e.g., toggle switch), dial, or various other types of the actuation mechanisms which may be operated by the user in order to change the diameter of the body 202. In certain embodiments, the hub 214 can include an opening 220 (e.g., slot, hole) that allows the actuator 218 to be moved such that the sheath 200 is controlled to switch between configurations to increase or decrease the size of the outer diameter 216 of the body 202.

As the actuator 218 is moved to the configuration of FIG. 3B, the actuator 218 pushes an additional length of the wire 206 out of the hub 214. Put another way, in the configuration shown in FIG. 3B, the overall length of the wire 206 outside the hub 214 is greater compared to the configuration of FIG. 3A. Because of the helical shape of the wire 206, the helical shape expands and causes the body 202 to expand as well. For example, because the overall length of the body 202 remains constant between the two configurations, when an additional length of the wire 206 is pushed out of the hub 214, the additional length of wire 206 will increase the size of the body 202. Because the shaft 204 comprises a flexible material, the body 202 can expand and become larger. In turn, the inner diameter and the outer diameter 216 of the body 202 increases and therefore creates additional space for a medical device to be inserted.

Although the wire 206 in FIGS. 3A and 3B is shown as one continuous wire, the body 202 could include multiple separate wires along the body 202. For example, different wires could be used in connection with different lengths of the body 202. In such examples, each wire could be separately controlled by separate actuators.

The support member 208 can extend longitudinally from the hub 214 to the distal end 212 and be designed to provide structural support and help with deliverability of an inserted medical device. The support member 208 can be embedded in the material of the shaft 204. In certain embodiments, multiple support members 208 are used in the body 202.

To reduce the size of the outer diameter 216 (and inner diameter) of the body 202, the movement of the actuator 218 can be reversed such that a portion of the wire 206 is pulled into the hub 214. As the length of the wire 206 outside the hub 214 is reduced, the wire 206 can be pulled radially inward to reduce the size of the outer diameter 216 (and inner diameter).

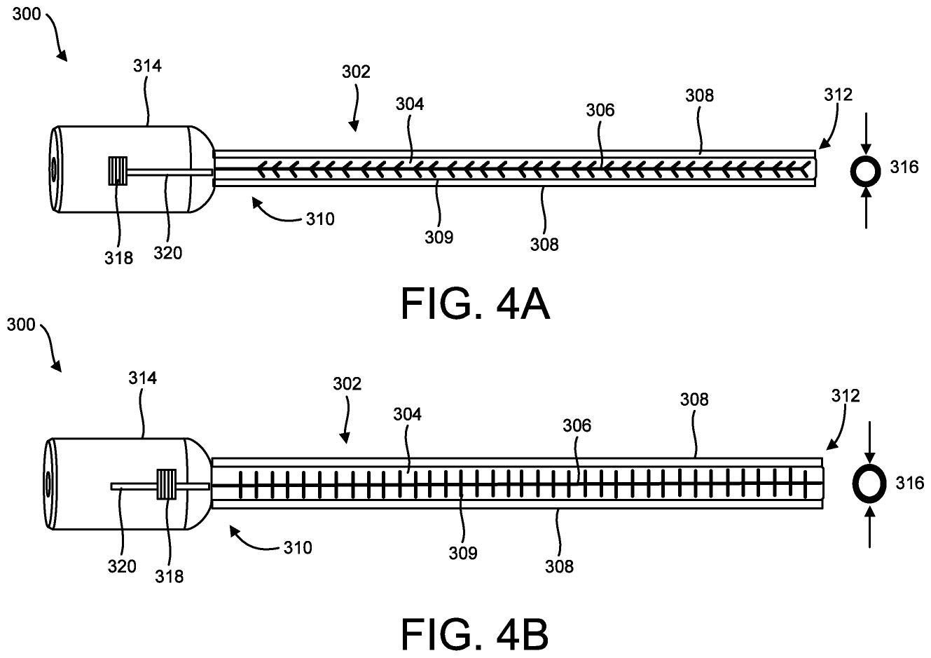

FIGS. 4A and 4B show a sheath 300 in different configurations. FIG. 4A shows the sheath 300 in a first configuration where the size of the outer diameter of the sheath 300 is smaller compared to the size of the outer diameter of the sheath 300 when it is in a second configuration, which is shown in FIG. 4B. In addition to being able to change the size of the outer diameter, the sheath 300 can be controlled to change the size of the inner diameter of the sheath 300 (e.g., the size of the diameter of a lumen within the sheath 300).

The sheath 300 includes a body 302, which includes a shaft 304, one or more wires 306, one or more support members 308, and multiple wing portions 309. The wing portions 309 are coupled between the wire 306 and the support members 308. In certain embodiments, the support members 308 and the wing portions 309 are made from a single part that is processed (e.g., machined, laser cut) to create the shape of the support members 308 and the wing portions 309. In certain embodiments, the wire 306 is also formed from the same part as the support members 308 and the wing portions 309. In certain embodiments, the body 302 includes one wire 306 and two support members 308. The shaft 304 extends between a proximal end 310 and a distal end 312, and the shaft 304 is coupled to a hub 314.

In certain embodiments, the wire 306, the support members 308, and/or the wing portions 309 are embedded in the shaft 304. For example, the shaft 304 could be molded around the wire 306, the support member 308, and/or the wing portions 309 such that the shaft 304 covers the wire 306, the support members 308, and/or the wing portions 309. As another example, the shaft 304 could comprise multiple layers (e.g., an inner shaft portion and an outer shaft portion), and the wire 306, the support members 308, and/or the wing portions 309 could be positioned between the layers.

The shaft 304 can comprise a first material, and the wire 306, the support members 308, and/or the wing portions 309 can comprise a second material. In some embodiments, the first material is a softer material (e.g., less rigid material) relative to the second material. In other words, the first material may have a higher flexibility and less resistance to deformation relative to the second material. Further, the first material may have a lower surface roughness relative to the second material such that the first material creates less friction when a medical device is inserted into the body 302. In some embodiments, the first material is or comprises an elastomer material (e.g., thermoplastic elastomer, thermoplastic polyurethane elastomer), and the second material is or comprises a metallic material such as steel (e.g., spring steel) or an alloy comprising nickel and titanium (e.g., nitinol). However, other applicable materials may be used in place of the materials mentioned above or in addition to such materials. In certain embodiments, a coating may be applied to an interior surface of the body 302 to lubricate the interior surface so that less friction is created between the body 302 and a medical device passing through the body 302. In embodiments where the shaft 304 includes multiple layers, the layers can comprise the same or different materials. Further, in certain embodiments, the wire 306, the support members 308, and/or the wing portions 309 could comprise different materials than each other.

The body 302 can be cylindrically shaped and have an inner diameter and an outer diameter 316—the size of which can be controlled to be decreased or increased. In the Figures, the inner diameter and the outer diameter 316 of the body 302 is smaller in FIG. 4A c compared to the inner diameter and outer diameter 316 in FIG. 4B.

The size of the body 302 in the radial direction can be modified using an actuator 318 that is coupled to the hub 314. The actuator 318 can be moved to cause the sheath 300 to switch between configurations. For example, the actuator 318 can be moved to increase or reduce the size of the inner diameter and the outer diameter 316. The actuator 318 may be a button, switch (e.g., toggle switch), dial, or various other types of the actuation mechanisms which may be operated by the user in order to change the diameter of the body 302. In certain embodiments, the hub 314 can include an opening 320 (e.g., slot, hole) that allows the actuator 318 to be moved such that the sheath 300 is controlled to switch between configurations to increase or decrease the size of the outer diameter 316 of the body 302.

As the actuator 318 is moved to the configuration of FIG. 4B, the actuator 318 pushes (or releases) part of the wire 306 outside the hub 314. Put another way, in the configuration shown in FIG. 4B, the overall length of the wire 306 outside the hub 314 is greater compared to the configuration of FIG. 4A. As the wire 306 is pushed (or released), the wing portions 309 move between a partially collapsed configuration to an expanded configuration. As shown in FIG. 4B, the wing portions 309 are less slanted or angled compared to their position shown in FIG. 4A. When the wing portions 309 move from the position of FIG. 4A to the position of FIG. 4B, the body 202 expands such that the size of the outer diameter 316 (and inner diameter) increase. Because the shaft 304 comprises a flexible material, the body 302 can expand and become larger. In turn, the inner diameter of the body 302 increases and therefore creates additional space for a medical device to be inserted.

Although the wire 306, the support members 308, and/or the wing portions 309 in FIGS. 4A and 4B could be manufactured as one continuous part, the body 302 could include multiple wires and wing portions along the body 302.

The support member 308 can extend longitudinally from the hub 314 to the distal end 312 and be designed to provide structural support and help with deliverability of an inserted medical device. The support member 308 can be embedded in the material of the shaft 304. In certain embodiments, multiple support members 308 are used in the body 302.

To reduce the size of the outer diameter 316 (and inner diameter), the movement of the actuator 318 can be reversed such that a portion of the wire 306 is pulled into the hub 314. As the actuator 318 is moved to the configuration of FIG. 4A, the actuator 318 pulls the wire 306 into the hub 314. Put another way, in the configuration shown in FIG. 4A, the overall length of the wire 306 outside the hub 314 is shorter compared to the configuration of FIG. 4B. As the wire 306 is pulled, the wing portions 309 are pulled such that they partially collapse. As shown in FIG. 4A, the wing portions 309 are slanted or angled compared to their position shown in FIG. 4B.

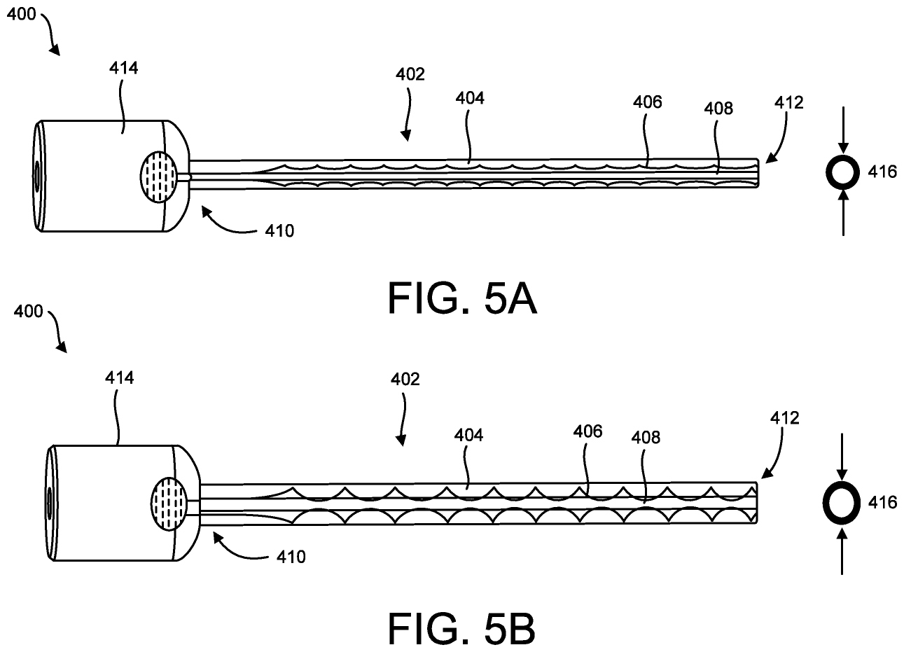

FIGS. 5A and 5B show a sheath 400 in different configurations, where the sizes of the outer diameter and inner diameter are increased or decreased passively. FIG. 5A shows the sheath 400 in a first configuration where the size of the outer diameter (and therefore inner diameter) of the sheath 400 is smaller compared to the size of the outer diameter of the sheath 400 when it is in a second configuration, which is shown in FIG. 5B.

The sheath 400 includes a body 402, which includes a shaft 404, one or more wires 406, and one or more support members 408. The shaft 404 extends between a proximal end 410 and a distal end 412, and the shaft 404 is coupled to a hub 414.

In certain embodiments, the wire 406 and/or the support member 408 is embedded in the shaft 404. For example, the shaft 404 could be molded around the wire 406 and/or the support member 408 such that the shaft 404 covers the wire 406 and/or the support member 408. As another example, the shaft 404 could comprise multiple layers (e.g., an inner shaft portion and an outer shaft portion), and the wire 406 and/or the support member 408 could be positioned between the layers.

The shaft 404 can comprise a first material, and the wire 406 and the support member 408 can comprise a second material. The first material is a softer material (e.g., less rigid material) relative to the second material. In other words, the first material may have a higher flexibility and less resistance to deformation relative to the second material. Further, the first material may have a lower surface roughness relative to the second material such that the first material creates less friction when a medical device is inserted into the body 402. In some embodiments, the first material is or comprises an elastomer material (e.g., thermoplastic elastomer, thermoplastic polyurethane elastomer), and the second material is or comprises a metallic material such as steel (e.g., spring steel) or an alloy comprising nickel and titanium (e.g., nitinol). However, other applicable materials may be used in place of the materials mentioned above or in addition to such materials. In certain embodiments, a coating may be applied to an interior surface of the body 402 to lubricate the interior surface so that less friction is created between the body 402 and a medical device passing through the body 402. In embodiments where the shaft 404 includes multiple layers, the layers can comprise the same or different materials. Further, in certain embodiments, the wire 406 and the support member 408 could comprise different materials than each other.

The body 402 can be cylindrically shaped and have an inner diameter and outer diameter 416—the size of which can be controlled to be decreased or increased. In the Figures, the inner diameter and the outer diameter 416 of the body 402 is smaller in FIG. 5A compared to the inner diameter and outer diameter 416 in FIG. 5B.

As a medical device (such as a blood pump) is inserted into the lumen defined by the body 402, the body 402 expands to a larger diameter size. After the medical device passes through a given section of the body 402, that section can then collapse to a smaller diameter. For example, the wire 406 can be arranged such that the wire 406 tightens or pulls the body 402 to a smaller diameter. As such, the default arrangement of the body 402 can be the smaller diameter arrangement, until a medical device expands sections of the body 402 as it passes through the lumen of the body 402. Because the shaft 404 comprises a flexible material, the body 402 can expand and become larger. In turn, the inner diameter of the body 402 increases and therefore creates additional space for a medical device to be inserted.

Although the wire 406 in FIGS. 5A and 5B is shown as one continuous wire, the body 402 could include multiple separate wires along the body 402. The support member 408 can extend longitudinally from the hub 414 to the distal end 412 and be designed to provide structural support and help with deliverability of an inserted medical device. The support member 408 can be embedded in the material of the shaft 404. In certain embodiments, multiple support members 408 are used in the body 402. In some embodiments, the wire 406 is coupled to or integrally formed with the support member 408.

Various modifications and additions can be made to the exemplary embodiments discussed without departing from the scope of the present disclosure. For example, while the embodiments described above refer to particular features, the scope of this disclosure also includes instances having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A sheath movable between a collapsed diameter and an expanded diameter, the sheath comprising:
a body having a lumen and movable between a first configuration and a second configuration, wherein the body has a first diameter size in the first configuration that is smaller than a second diameter size in the second configuration, wherein the body comprises a shaft made from a first material and a wire comprising a second material.

2. The sheath of claim 1, further comprising:
a hub coupled to the body; and
an actuator coupled to the hub, wherein—upon actuation—the actuator is configured to cause the body to move between the first configuration and the second configuration.

3. The sheath of claim 2, wherein—upon actuation—the actuator is configured to pull the wire into the hub to cause the body to move from the second diameter size to the first diameter size.

4. The sheath of claim 3, wherein the actuator is a switch movable within a slot in the hub.

5. The sheath of claim 1, wherein the wire is helical shaped.

6. The sheath of claim 1, wherein the wire is coupled to or integral with wing portions that are collapsible in response to the wire being pulled.

7. The sheath of claim 1, wherein the wire is embedded in the shaft.

8. The sheath of claim 1, wherein the first material comprises an elastomer, wherein the second material comprises a metal.

9. The sheath of claim 1, wherein the first material comprises a thermoplastic polyurethane elastomer.

10. The sheath of claim 1, further comprising:
a support member coupled to or formed as part of the body and extending along a longitudinal axis of the body.

11. The sheath of claim 10, further comprising wing portions positioned between the wire and the support member.

12. The sheath of claim 11, wherein the wing portions and the wire are integrally formed from one part.

13. The sheath of claim 10, wherein the wing portions are arranged perpendicular to the support member in the second configuration and are arranged at an angle less than 90 degrees in the first configuration.

14. The sheath of claim 1, wherein the first material has a lower durometer value compared to the second material.

15. The sheath of claim 1, wherein the body expands to the second diameter size as a medical device is inserted through the lumen.

16. The sheath of claim 15, wherein the wire is arranged to collapse before and after insertion of the medical device to collapse the body to the first diameter size.

17. The sheath of claim 1, wherein the first diameter size and the second diameter size are inner diameters of the body defined by the lumen.

18. The sheath of claim 1, wherein the first diameter size and the second diameter size are outer diameters of the body defined by an outer surface of the body.

19. A sheath movable between a collapsed diameter and an expanded diameter, the sheath comprising:
a body having a lumen and means for actuating the body between the collapsed diameter and the expanded diameter.

20. The sheath of claim 19, wherein the means for actuating comprises a wire and an actuator coupled to the wire and configured to pull the wire to actuate the body to the collapsed diameter.
